(19) United States
(12) Patent Application Publication
(10) Pub. No.: US 2021/0303802 A1
(43) Pub. Date: Sep. 30, 2021

(54) PROGRAM STORAGE MEDIUM, INFORMATION PROCESSING APPARATUS AND METHOD FOR ENCODING SENTENCE

(71) Applicant: FUJITSU LIMITED, Kawasaki-shi (JP)

(72) Inventor: Hajime Morita, Kawasaki (JP)

(73) Assignee: FUJITSU LIMITED, Kawasaki-shi (JP)

(21) Appl. No.: 17/206,188

(22) Filed: Mar. 19, 2021

(30) Foreign Application Priority Data
Mar. 26, 2020 (JP) 2020-056889

(51) Int. Cl. G06F 40/47; G06F 16/31; G06N 20/00; G06N 5/04; G06F 40/289

(52) U.S. Cl. CPC G06F 40/47; G06F 16/322; G06F 40/289; G06N 5/04; G06N 20/00

(57) ABSTRACT

A sentence is vectorized and encoded for further being processed by a computer. The encoding process includes, identifying a common ancestor node of a first node corresponding to a first segment in a sentence and a second node corresponding to a second segment in the sentence, the first node and the second node being included in a dependency tree generated based on the sentence, acquiring a vector of the common ancestor node by encoding each node included in the dependency tree in accordance with a path from each of leaf nodes included in the dependency tree to the common ancestor node, and encoding, based on the vector of the common ancestor node, each of nodes included in the dependency tree in accordance with the path from the common ancestor node to the leaf nodes.

### CROSS-REFERENCE TO RELATED APPLICATION

This application is based upon and claims the benefit of priority of the prior Japanese Patent Application No. 2020-56889, filed on Mar. 26, 2020, the entire contents of which are incorporated herein by reference.

### FIELD

The embodiments discussed herein are related to a technology for encoding a sentence or a word.

### BACKGROUND

In natural language processing, a sentence or a word (segment) in a sentence is often vectorized before it is processed. It is important to generate a vector, containing a feature of a sentence or a word, well.

It has been known that a sentence or a word (segment) is vectorized by, for example, a long short-term memory (LSTM) network. The LSTM network is a recursive neural network that may hold information on a word as a vector chronologically and generate a vector of the word by using the held information.

It has been known that a sentence or a word is vectorized by, for example, a tree-structured LSTM network (see Kai Sheng Tal et al, “Improved Semantic Representations From Tree-Structured Long Short-Term Memory Networks”, PP. 1556-1566, Association for Computational Linguistics, Jul. 26-31, 2015, for example). The tree-structured LSTM network is acquired by generalizing a chain-structured LSTM network to a tree-structured network topology. FIG. 12 is a reference diagram illustrating an LSTM network. The diagram on the upper side of FIG. 12 illustrates a chain-structured LSTM network. For example, an LSTM to which a word “x1” is input generates a vector “y” of the input word “x1”. An LSTM to which a word “x2” is input generates a vector “y2” of the word “x2” by also using the vector “y1” of the previous word “x1”. The diagram on the lower side of FIG. 12 illustrates a tree-structured LSTM network including arbitrary branching factors.

A technology has been known that utilizes a dependency tree that represents a dependency between words in a sentence by using a tree-structured LSTM network (hereinafter, an LSTM network is called “LSTM”). For example, a technology has been known that extracts a relation between words in a sentence by using information on the entire structure of a dependency tree for the sentence (see Miwa et al, “End-To-End Relation Extraction using LSTMs on Sequences and Tree Structures”, PP. 1105-1116, Association for Computational Linguistics, Aug. 7-12, 2016, for example).

### SUMMARY

According to an aspect of the embodiments, a method for encoding a sentence includes: identifying a common ancestor node of a first node corresponding to a first segment in a sentence and a second node corresponding to a second segment in the sentence, the first node and the second node being included in a dependency tree generated based on the sentence; acquiring a vector of the common ancestor node by encoding each node included in the dependency tree in accordance with a path from each of leaf nodes included in the dependency tree to the common ancestor node; and encoding, based on the vector of the common ancestor node, each of nodes included in the dependency tree in accordance with the path from the common ancestor node to the leaf nodes.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a functional block diagram illustrating a configuration of a machine learning device according to Embodiment 1;

FIG. 2 is a functional block diagram illustrating a configuration of a prediction device according to Embodiment 1;

FIG. 3 illustrates an example of dependencies in a sentence;

FIG. 4 illustrates an example of tree-structured encoding according to Embodiment 1;

FIG. 5 illustrates an example of a flowchart of relation extraction and learning processing according to Embodiment 1;

FIG. 6 illustrates an example of the relation extraction and learning processing according to Embodiment 1;

FIG. 7 illustrates an example of a flowchart of relation extraction and prediction processing according to Embodiment 1;

FIG. 8 is a functional block diagram illustrating a configuration of a machine learning device according to Embodiment 2;

FIG. 9 is a functional block diagram illustrating a configuration of a prediction device according to Embodiment 2;

FIG. 10 illustrates an example of tree-structured encoding according to Embodiment 2;

FIG. 11 illustrates an example of a computer that executes an encoding program;

FIG. 12 is a reference diagram illustrating an LSTM network; and

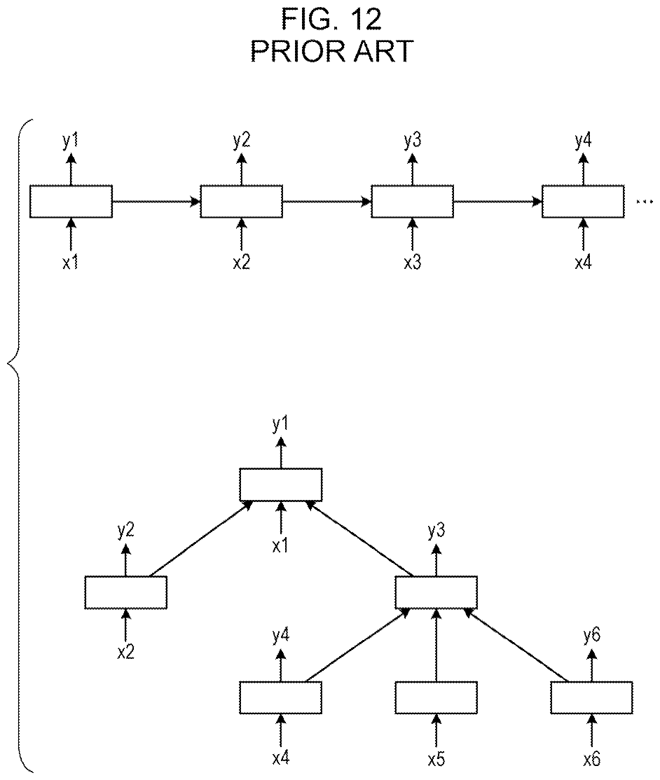

FIG. 13 illustrates a reference example of encoding on a representation outside an SP.

### DESCRIPTION OF EMBODIMENTS

For example, from a sentence “Medicine A was dosed to a randomly selected disease B patient, then, was found effective”, a relation (effective) between “Medicine A” and “disease B” may be extracted (determined). According to such a technology, with respect to a sentence, word-level information is encoded in an LSTM, and dependency-tree-level information with a shortest dependency path (shortest path: SP) only is encoded in a tree-structured LSTM to extract a relation. The term “SP” refers to the shortest path of dependency between words the relation of which is to be extracted and is a path between “Medicine A” and “disease B” in the sentence above. From an experiment with focus on the extraction of a relation, a better result was acquired when a dependency tree only with SP was used than a case where the entire dependency tree for a sentence was used.

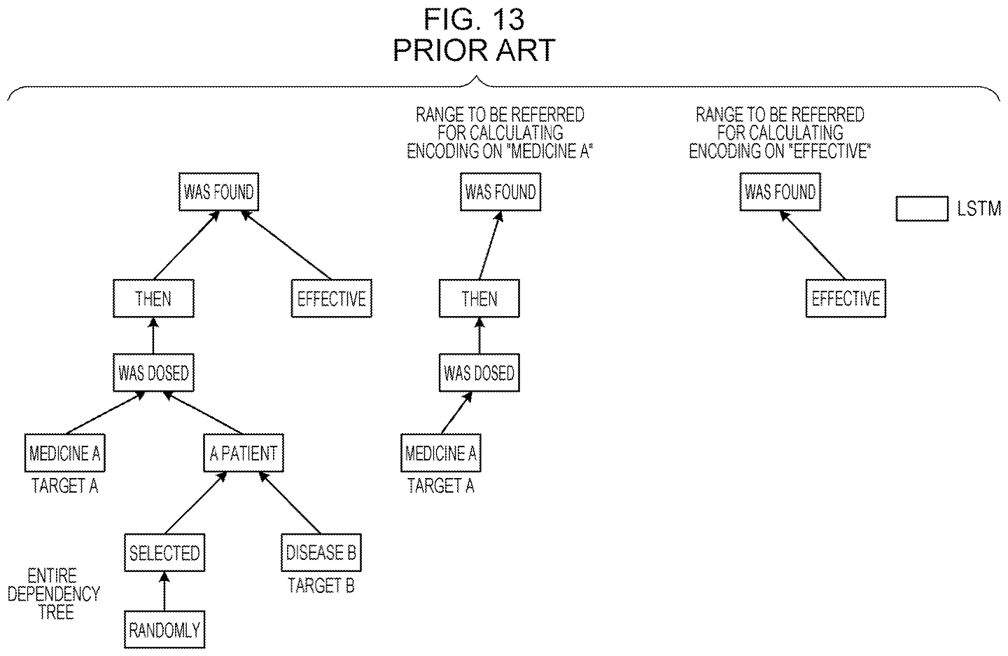

Even by using the entire dependency tree for a sentence or even by using a dependency tree with the shortest dependency path only, it is difficult to utilize information within the SP for encoding a representation outside the SP. The difficulty of use of information within the SP for encoding a representation outside the SP will be described with reference to FIG. 13. FIG. 13 illustrates a reference example of encoding on a representation outside an SP. Suppose a case where, from the above-described sentence “Medicine A was dosed to a randomly selected disease B patient, then, was found effective”, a relation (“effective”) between “Medicine A” and “disease B” is to be extracted (determined).

As illustrated in FIG. 13, the left diagram illustrates an entire dependency tree. Each of rectangular boxes represents an LSTM. SP refers to a path between “Medicine A” and “disease B”. The tree structure in the middle diagram represents a range to be referred for calculating encoding on “Medicine A”. The tree structure in the right diagram is a range to be referred for calculating encoding on “effective” representing the relation.

Under this condition, in the entire dependency tree, because encoding is performed along a structure of the entire dependency tree for the sentence, it is difficult to encode a word outside the SP, for example, a word without a dependency relation with the SP by using a word within the SP. For example, in FIG. 13, “effective” representing the relation is a representation outside the SP. The range to be referred for encoding the word “effective” outside the SP, for example, without the dependency relation is “was found” only, and the encoding may not be performed by using a feature of, for example, the word “Medicine A” within the SP under “was found”. For example, it is difficult to determine the importance of the representation outside the SP in the dependency tree.

Even when the dependency tree having the SP only is used, it is still difficult to use information within the SP for encoding a representation outside the SP, like the case where the entire dependency tree is used.

As a result, when an important representation indicating a relation is outside the SP, it is difficult to extract the relation between words within the SP. Therefore, disadvantageously, the sentence may not be encoded based on outside of the SP of the dependency tree.

Hereinafter, embodiments of an encoding program, an information processing apparatus, and an encoding method disclosed in the present application will be described in detail with reference to the drawings. According to the embodiments, a machine learning device and a prediction device will separately be described as the information processing apparatus. Note that the present disclosure is not limited by the embodiments.

### Embodiment 1

[Configuration of Machine Learning Device]

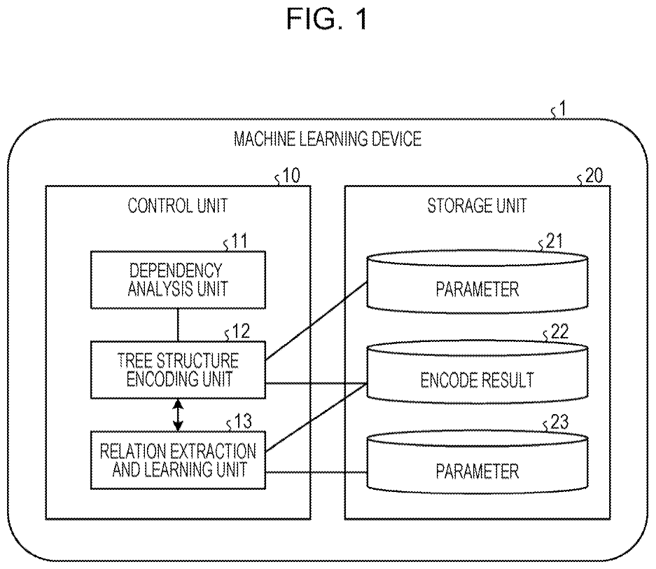

FIG. 1 is a functional block diagram illustrating a configuration of a machine learning device according to an embodiment. A machine learning device 1 aggregates information of an entire sentence to a common ancestor node in a dependency tree of the entire sentence and encodes each node of the dependency tree by using the aggregated information. By using the encoding result, the machine learning device 1 learns a relation between a first segment and a second segment included in the sentence. The term “dependency tree” refers to dependencies between words in a sentence represented by a tree-structured LSTM network. Hereinafter, the LSTM network is called “LSTM”. The segment may also be called a “word”.

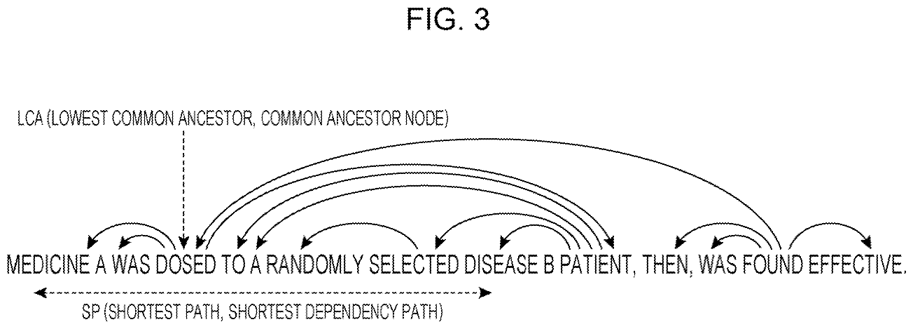

An example of dependencies in a sentence will be described with reference to FIG. 3. FIG. 3 illustrates an example of dependencies in a sentence. As illustrated in FIG. 3, a sentence “Medicine A was dosed to a randomly selected disease B patient, then, was found effective” is given. The sentence is divided into sequences in units of segment, “Medicine A”, “was”, “dosed”, “to”, “a”, “randomly”, “selected”, “disease B”, “patient”, “then”, “was”, “found”, and “effective”.

The dependency of “Medicine A” is “dosed”. The dependency of “randomly” is “selected”. The dependency of “selected” and “disease B” is “patient”. The dependency of “patient” is “dosed”. The dependency of “dosed” is “then”. The dependency of “then” and “effective” is “found”.

In order to extract (determine) the relation (“effective”) between “Medicine A” and “disease B”, the path between “Medicine A” and “disease B” is the shortest dependency path (shortest path: SP). The term “SP” refers to the shortest path of dependency between the word “Medicine A” and the word “disease B” the relation of which is to be extracted and is the path between “Medicine A” and “disease B” in the sentence above. The word “effective” representing the relation is outside of the SP in the sentence.

“dosed” is a common ancestor node (lowest common ancestor: LCA) of “Medicine A” and “disease B”.

Referring back to FIG. 1, the machine learning device 1 has a control unit 10 and a storage unit 20. The control unit 10 is implemented by an electronic circuit such as a central processing unit (CPU). The control unit 10 has a dependency analysis unit 11, a tree structure encoding unit 12, and a relation extraction and learning unit 13. The tree structure encoding unit 12 is an example of an identification unit, a first encoding unit and a second encoding unit.

The storage unit 20 is implemented by, for example, a semiconductor memory device such as a random-access memory (RAM) or a flash memory, a hard disk, an optical disk, or the like. The storage unit 20 has a parameter 21, an encode result 22 and a parameter 23.

The parameter 21 is a kind of parameter to be used by an LSTM for each word in a word sequence of a sentence for encoding the word by using a tree-structured LSTM (tree LSTM). One LSTM encodes one word by using the parameter 21. The parameter 21 includes, for example, a direction of encoding. The term “direction of encoding” refers to a direction from a word having the nearest word vector to a certain word when the certain word is to be encoded. The direction of encoding may be, for example, “above” or “below”.

The encode result 22 represents an encode result (vector) of each word and an encode result (vector) of a sentence. The encode result 22 is calculated by the tree structure encoding unit 12.

The parameter 23 is a parameter to be used for learning a relation between words by using the encode result 22. The parameter 23 is used and is properly corrected by the relation extraction and learning unit 13.

The dependency analysis unit 11 analyzes a dependency in a sentence. For example, the dependency analysis unit 11 performs morphological analysis on a sentence and divides the sentence into sequences of morphemes (in units of segment). The dependency analysis unit 11 performs dependency analysis in units of segment on the divided sequences. The dependency analysis may use any parsing tool.

The tree structure encoding unit 12 encodes each segment by using the tree-structured LSTM of a tree converted to have a tree structure including dependencies of segments. For example, the tree structure encoding unit 12 uses dependencies of segments analyzed by the dependency analysis unit 11 and converts them to a dependency tree having a tree structure including the dependencies of the segments. For a first segment and a second segment included in a sentence, the tree structure encoding unit 12 identifies a common ancestor node (LCA) of a first node corresponding to the first segment and a second node corresponding to the second segment, which are two nodes included in the converted dependency tree. The tree structure encoding unit 12 encodes each node included in the dependency tree along a path from each of leaf nodes included in the dependency tree to the LCA by using the parameter 21 and thus acquires a vector being an encoding result of the LCA. For example, the tree structure encoding unit 12 acquires the encoding result vector of the LCA by aggregating information of the nodes to the LCA along the path from each of leaf nodes to the LCA. Based on the encoding result vector of the LCA, the tree structure encoding unit 12 encodes each of the nodes included in the dependency tree along the path from the LCA to the leaf nodes by using the parameter 21. For example, the tree structure encoding unit 12 aggregates information of the entire sentence to the LCA and then causes the aggregated information to reversely propagate to encode each node of the dependency tree.

By using the encoding result vectors of the nodes, the tree structure encoding unit 12 acquires a vector of the sentence.

When the vector of the sentence and a relation label (correct answer label) that is already known are input to the relation extraction and learning unit 13, the relation extraction and learning unit 13 learns a machine learning model such that a relation label corresponding to the relation between the first segment and the second segment included in the sentence is matched with the input relation label. For example, when a vector of a sentence is input to the machine learning model, the relation extraction and learning unit 13 outputs a relation between a first segment and a second segment included in the sentence by using the parameter 23. If the relation label corresponding to the output relation is not matched with the already known relation label (correct answer label), the relation extraction and learning unit 13 causes the tree structure encoding unit 12 to reversely propagate the error of the information. The relation extraction and learning unit 13 learns the machine learning model by using the vectors of the nodes corrected with the error and the corrected parameter 23. For example, the relation extraction and learning unit 13 receives input of the vector of a sentence and a correct answer label corresponding to the vector of the sentence and updates the machine learning model through machine learning based on a difference between a prediction result corresponding to the relation between the first segment and the second segment included in the sentence to be output by the machine learning model in accordance with the input and the correct answer label.

As the machine learning model, a neural network (NN) or a support vector machine (SVM) may be adopted. For example, the NN may be a convolutional neural network (CNN) or a recurrent neural network (RNN). The machine learning model may be, for example, a machine learning model implemented by a combination of a plurality of machine learning models such as a machine learning model implemented by a combination of a CNN and an RNN.

[Configuration of Prediction Device]

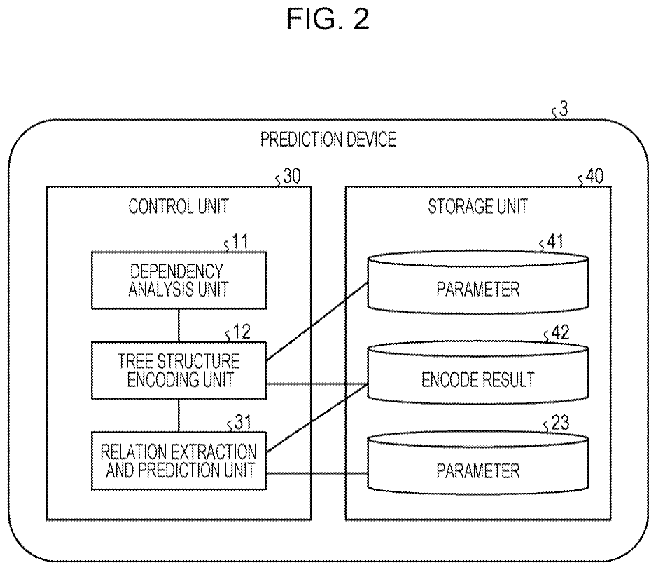

FIG. 2 is a functional block diagram illustrating a configuration of a prediction device according to Embodiment 1. A prediction device 3 aggregates information of an entire sentence to a common ancestor node in a dependency tree of the entire sentence and encodes each node of the dependency tree by using the aggregated information. By using the encoding result, the prediction device 3 predicts a relation between a first segment and a second segment included in the sentence.

Uke the one in FIG. 1, the prediction device 3 has a control unit 30 and a storage unit 40. The control unit 30 is implemented by an electronic circuit such as a central processing unit (CPU). The control unit 30 has a dependency analysis unit 11, a tree structure encoding unit 12, and a relation extraction and prediction unit 31. Because the dependency analysis unit 11 and the tree structure encoding unit 12 have the same configurations as those in the machine learning device 1 illustrated in FIG. 1, like numbers refer to like parts, and repetitive description on the configurations and operations are omitted. The tree structure encoding unit 12 is an example of the identification unit, the first encoding unit and the second encoding unit.

The storage unit 40 is implemented by, for example, a semiconductor memory device such as a RAM or a flash memory, a hard disk, an optical disk, or the like. The storage unit 40 has a parameter 41, an encode result 42 and a parameter 23.

The parameter 41 is a parameter to be used by an LSTM for each word in word sequences of a sentence for encoding the word by using a tree-structured LSTM. One LSTM encodes one word by using the parameter 41. The parameter 41 includes, for example, a direction of encoding. The term “direction of encoding” refers to a direction from a word having a word vector before used to a certain word when the certain word is to be encoded. The direction of encoding may be, for example, “above” or “below”. The parameter 41 corresponds to the parameter 21 in the machine learning device 1.

The encode result 42 represents an encode result (vector) of each word and an encode result (vector) of a sentence. The encode result 42 is calculated by the tree structure encoding unit 12. The encode result 42 corresponds to the encode result 22 in the machine learning device 1.

The parameter 23 is a parameter to be used for predicting a relation between words by using the encode result 42. The same parameter as the parameter 23 optimized by the machine learning in the machine learning device 1 is applied to the parameter 23.

When a vector of a sentence is input to the learned machine learning model, the relation extraction and prediction unit 31 predicts a relation between a first segment and a second segment included in the sentence. For example, when a vector of a sentence is input to the learned machine learning model, the relation extraction and prediction unit 31 predicts a relation between a first segment and a second segment included in the sentence by using the parameter 23. The relation extraction and prediction unit 31 outputs a relation label corresponding to the predicted relation. The learned machine learning model is the one that has learned by the relation extraction and learning unit 13 in the machine learning device 1.

[Example of Tree-Structured Encoding]

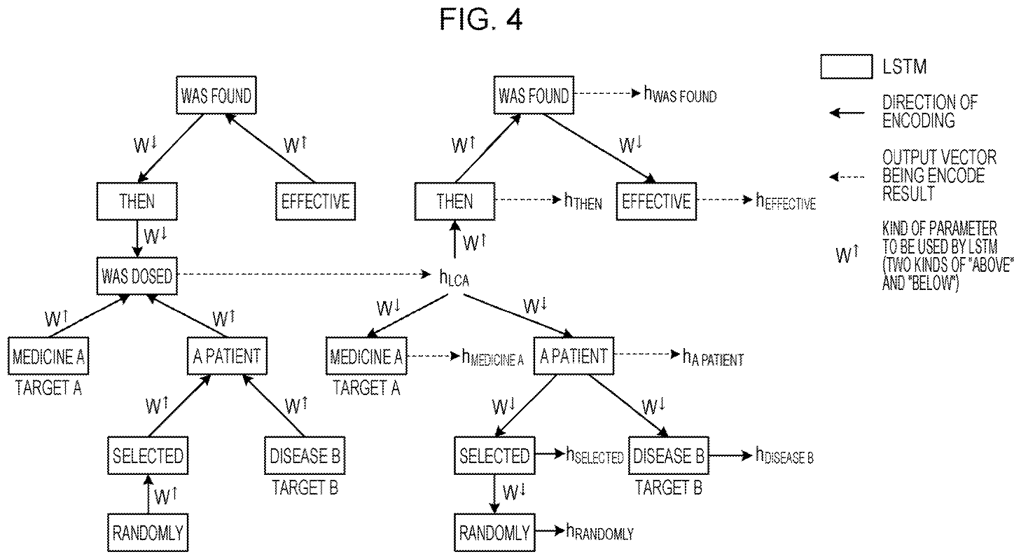

FIG. 4 illustrates an example of tree-structured encoding according to Embodiment 1. Suppose a case where a sentence “Medicine A was dosed to a randomly selected disease B patient, then, was found effective” is given and a relation (effective) between “Medicine A” and “disease B” is to be extracted (determined).

The left diagram of FIG. 4 illustrates a converted dependency tree of the sentence. The tree is converted by the tree structure encoding unit 12. For example, the tree structure encoding unit 12 uses dependencies of segments in the sentence analyzed by the dependency analysis unit 11 and converts them to a converted dependency tree having a tree structure including the dependencies of the segments. Each of rectangular boxes in FIG. 4 represents an LSTM.

For “Medicine A” and “disease B” included in the sentence, the tree structure encoding unit 12 identifies a common ancestor node (LCA) of a node corresponding to “Medicine A” and a node corresponding to “disease B”, which are two nodes included in the converted dependency tree. The identified LCA is a node corresponding to “was dosed”.

The tree structure encoding unit 12 encodes each node included in the converted dependency tree along a path from each of leaf nodes included in the converted dependency tree to the LCA by using the parameter 21 and thus acquires a vector being an encoding result of the LCA. For example, the tree structure encoding unit 12 aggregates information of the nodes to the LCA along the path from each of leaf nodes to the LCA. In the left diagram, the nodes corresponding to “Medicine A”, “randomly”, “disease B”, and “effective” are the leaf nodes.

As illustrated in the left diagram, the tree structure encoding unit 12 inputs “Medicine A” to the LSTM. The tree structure encoding unit 12 outputs an encode result (vector) encoded by the LSTM to the LSTM of “was dosed” (LCA) positioned “above” indicated by the parameter.

The tree structure encoding unit 12 inputs “randomly” to the LSTM. The tree structure encoding unit 12 outputs the encode result (vector) encoded by the LSTM to the LSTM of “selected” positioned “above” indicated by the parameter. The tree structure encoding unit 12 inputs “selected” and the vector from “randomly” to the LSTM. The tree structure encoding unit 12 outputs the encode result (vector) encoded by the LSTM to the LSTM of “a patient” positioned “above” indicated by the parameter.

The tree structure encoding unit 12 inputs “disease B” to the LSTM. The tree structure encoding unit 12 outputs the encode result (vector) encoded by the LSTM to the LSTM of “a patient” positioned “above” indicated by the parameter. The tree structure encoding unit 12 inputs “a patient” and the vectors from “selected” and “disease B” to the LSTM. The tree structure encoding unit 12 outputs the encode result (vector) encoded by the LSTM to the LSTM of “was dosed” (LCA) positioned “above” indicated by the parameter.

On the other hand, the tree structure encoding unit 12 inputs “effective” to the LSTM. The tree structure encoding unit 12 outputs the encode result (vector) encoded by the LSTM to the LSTM of “was found” positioned “above” indicated by the parameter. The tree structure encoding unit 12 inputs “was found” and the vector from “effective” to the LSTM. The tree structure encoding unit 12 outputs the encode result (vector) encoded by the LSTM to the LSTM of “then” positioned “below” indicated by the parameter.

The tree structure encoding unit 12 inputs “then” and the vector from “was found” to the LSTM. The tree structure encoding unit 12 outputs the encode result (vector) encoded by the LSTM to the LSTM of “dosed” (LCA) positioned “below” indicated by the parameter.

The tree structure encoding unit 12 inputs “was dosed” and the encode results (vectors) of “Medicine A”, “a patient”, and “then” to the LSTM. The tree structure encoding unit 12 acquires the encode result (vector) that has been encoded. For example, the tree structure encoding unit 12 aggregates information of the nodes to the LCA along the path from each of leaf nodes to the LCA.

After that, based on the encode result (vector) of the LCA, the tree structure encoding unit 12 encodes each of the nodes included in the dependency tree along the path from the LCA to the leaf nodes by using the parameter 21. For example, the tree structure encoding unit 12 aggregates information of the entire sentence to the LCA and then causes the aggregated information to reversely propagate to encode each node of the converted dependency tree.

As illustrated in the right diagram, suppose that the encode result (vector) of LCA is h_{LCA}. The tree structure encoding unit 12 outputs h_{LCA} to the LSTMs of “Medicine A” and “a patient” positioned “below” indicated by the parameters toward the leaf nodes. The tree structure encoding unit 12 outputs h_{L}ca to the LSTM of “then” positioned “above” indicated by the parameter toward the leaf node.

The tree structure encoding unit 12 inputs “Medicine A” and h_{LCA} to the LSTM. The tree structure encoding unit 12 outputs h_{Medicine A} as the encode result (vector) encoded by the LSTM.

The tree structure encoding unit 12 inputs “a patient” and h_{LCA} to the LSTM. The tree structure encoding unit 12 outputs h_{a patient} as the encode result (vector) encoded by the LSTM. The tree structure encoding unit 12 outputs h_{a patient} to the LSTMs of “selected” and “disease B” positioned “below” indicated by the parameters toward the leaf nodes.

The tree structure encoding unit 12 inputs “disease B” and the vector from “a patient” to the LSTM. The tree structure encoding unit 12 outputs h_{disease B} as the encode result (vector) encoded by the LSTM. The tree structure encoding unit 12 inputs “selected” and the vector from “patient” to the LSTM. The tree structure encoding unit 12 outputs h_{selected} as the encode result (vector) encoded by the LSTM. The tree structure encoding unit 12 outputs h_{selected} to the LSTM of “randomly” positioned “below” indicated by the parameter toward the leaf node.

The tree structure encoding unit 12 inputs “randomly” and the vector from “selected” to the LSTM. The tree structure encoding unit 12 outputs h_{randomly} as the encode result (vector) encoded by the LSTM.

On the other hand, the tree structure encoding unit 12 inputs “then” and h_{LCA} to the LSTM. The tree structure encoding unit 12 outputs hₜₕₑₙ as the encode result (vector) encoded by the LSTM. The tree structure encoding unit 12 outputs hₜₕₑₙ to the LSTM of “was found” positioned “above” indicated by the parameter toward the leaf node.

The tree structure encoding unit 12 inputs “was found” and the vector from “then” to the LSTM. The tree structure encoding unit 12 outputs h_{was} round as the encode result (vector) encoded by the LSTM. The tree structure encoding unit 12 outputs h_{was found} to the LSTM of “effective” positioned “below” indicated by the parameter toward the leaf node.

The tree structure encoding unit 12 inputs “effective” and the vector from “was found” to the LSTM. The tree structure encoding unit 12 outputs h_{effective} as the encode result (vector) encoded by the LSTM.

By using the vectors representing the encode results of the nodes, the tree structure encoding unit 12 acquires a vector of the sentence. The tree structure encoding unit 12 may acquire a vector h_{sentence} of the sentence as follows. h_{sentence}=[h_{Medicine A};h_{randomly};h_{selected};h_{disease B};h_{a patient};h_{was dosed};hₜₕₑₙ;h_{effective};h_{was found};]

Thus, the tree structure encoding unit 12 may encode the sentence based on outside of the SP of “Medicine A” and “disease B” in the dependency tree. For example, the tree structure encoding unit 12 may encode the sentence not only based on the SP of the “Medicine A” and “disease B” in the dependency tree but also based on the outside of the SP because information on the nodes including “effective” representing a relation that exists outside the SP is also gathered to the LCA. As a result, the relation extraction and learning unit 13 may generate a highly-precise machine learning model to be used for extracting a relation between words. In addition, the relation extraction and prediction unit 31 may extract a relation between words with high precision by using the machine learning model.

[Flowchart of Relation Extraction and Learning Processing]

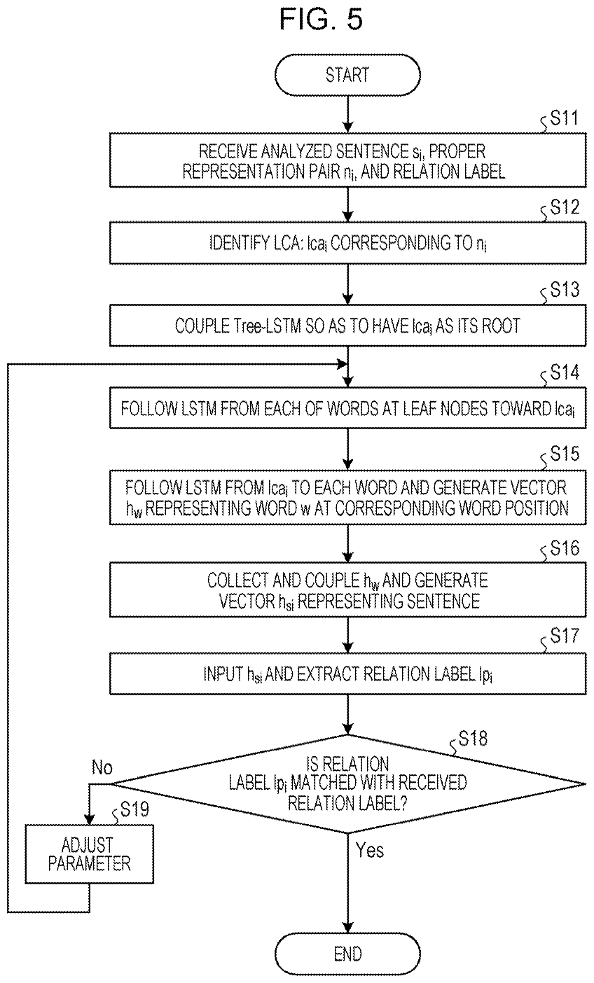

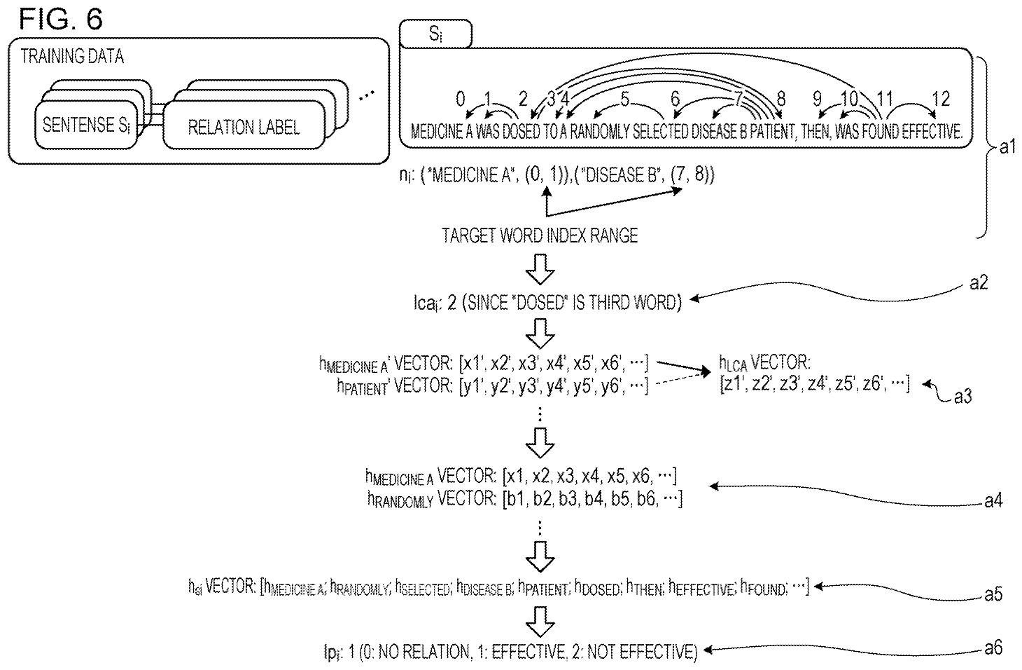

FIG. 5 illustrates an example of a flowchart of relation extraction and learning processing according to Embodiment 1. The example of the flowchart will be described properly with reference to an example of relation extraction and learning processing according to Embodiment 1 illustrated in FIG. 6.

The tree structure encoding unit 12 receives a sentence sᵢ analyzed by the dependency analysis, a proper representation pair nᵢ, and an already known relation label (step S11). As indicated by reference “a1” in FIG. 6, a sentence sᵢ “Medicine A was dosed to a randomly selected disease B patient, then, was found effective” and a proper representation pair “Medicine A” and “disease B” are given. In the sentence sᵢ, dependencies between words are analyzed. The proper representation pair is a pair of words that are targets the relation of which is to be learned. A range of an index in the sentence is indicated for each of the words. The index is information indicating at what place the word exists in the sentence. The index is counted from 0. “Medicine A” is between 0 and 1. “disease B” is between 7 and 8. The proper representation pair nᵢ corresponds to the first segment and the second segment.

The tree structure encoding unit 12 identifies Icaᵢ as LCA (common ancestor node) corresponding to the proper representation pair nᵢ (step S12). As indicated by reference “a2” in FIG. 6, the index Icaᵢ of the common ancestor node is “2”. For example, the third “dosed” is the word of LCA.

The tree structure encoding unit 12 couples the LSTMs in a tree structure having Icaᵢ as its root (step S13). For example, the tree structure encoding unit 12 uses dependencies of the segments and forms a converted dependency tree having a tree structure including the dependencies of the segments.

The tree structure encoding unit 12 follows the LSTMs from each of the words at the leaf nodes toward Icaᵢ (step S14). As indicated by reference “a3” in FIG. 6, for example, an encode result vector h_{LCA}′ of the LCA is acquired from the vector h_{medicine A}′ of “Medicine A”, the vector hₚₐₜᵢₑₙₜ′ of “patient”, and the vectors of other words. For example, the tree structure encoding unit 12 acquires the encoding result vector of the LCA by aggregating information of the nodes to the LCA along the path from each of leaf nodes to the LCA.

The tree structure encoding unit 12 follows the LSTMs from Icaᵢ to each of the words and generates a vector h_{w} representing a certain word w at the corresponding word position (step S15). As indicated by reference “a4” in FIG. 6, for example, a vector h_{medicine A} of “Medicine A” and a vector h_{randomly} of “randomly” are generated. For example, the tree structure encoding unit 12 aggregates information of the entire sentence to the LCA and then causes the aggregated information to reversely propagate to encode each node of the converted dependency tree.

The tree structure encoding unit 12 collects and couples the vectors h_{w} of the words and generates a vector hₛᵢ representing the sentence (step S16). As indicated by reference “a5” in FIG. 6, the vector h_{Medicine A} of “Medicine A”, the vector h_{randomly} of “randomly”, . . . are collected and are coupled to generate the vector hₛᵢ of the sentence sᵢ.

The relation extraction and learning unit 13 inputs the vector hₛᵢ of the sentence to the machine learning model and extracts a relation label Ipᵢ (step S17). As indicated by reference “a6” in FIG. 6, the relation extraction and learning unit 13 extracts the relation label Iₚᵢ. One of “0” indicating no relation, “1” indicating related and effective, and “2” indicating related but not effective is extracted. The relation extraction and learning unit 13 determines whether the relation label Ipᵢ is matched with the received relation label or not (step S18). If it is determined that the relation label Ipᵢ is not matched with the received relation label (No in step S18), the relation extraction and learning unit 13 adjusts the parameter 21 and the parameter 23 (step S19). The relation extraction and learning unit 13 moves to step S14 for further learning.

On the other hand, if the relation label Ipᵢ is matched with the received relation label (Yes in step S18), the relation extraction and learning unit 13 exits the relation extraction and learning processing.

[Flowchart of Relation Extraction and Prediction Processing]

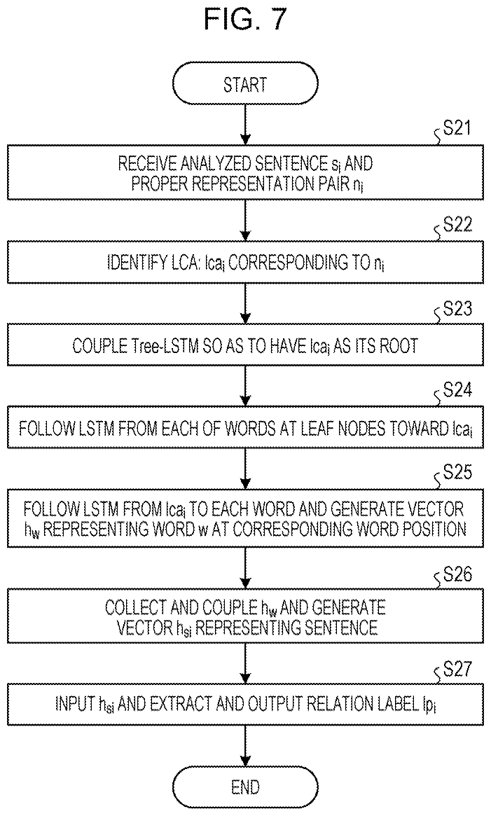

FIG. 7 illustrates an example of a flowchart of relation extraction and prediction processing according to Embodiment 1. The tree structure encoding unit 12 receives a sentence sᵢ analyzed by the dependency analysis and a proper representation pair nᵢ (step S21). The tree structure encoding unit 12 identifies Icaᵢ as the LCA (common ancestor node) corresponding to the proper representation pair nᵢ (step S22).

The tree structure encoding unit 12 couples the LSTMs in a tree structure having Icaᵢ as its root (step S23). For example, the tree structure encoding unit 12 uses dependencies of the segments and forms a converted dependency tree having a tree structure including the dependencies of the segments.

The tree structure encoding unit 12 follows the LSTMs from each of the words at the leaf nodes toward Icaᵢ (step S24). For example, the tree structure encoding unit 12 acquires the encoding result vector of the LCA by aggregating information of the nodes to the LCA along the path from each of leaf nodes to the LCA.

The tree structure encoding unit 12 follows the LSTMs from Icaᵢ to each of the words and generates a vector h_{w} representing a certain word w at the corresponding word position (step S25). For example, the tree structure encoding unit 12 aggregates information of the entire sentence to the LCA and then causes the aggregated information to reversely propagate to encode each node of the converted dependency tree.

The tree structure encoding unit 12 collects and couples the vectors h_{w} of the words and generates a vector hₛᵢ representing the sentence (step S26). The relation extraction and prediction unit 33 inputs the vector hₛᵢ of the sentence to the machine learning model that has learned, extracts a relation label Ipᵢ and outputs the extracted relation label Ipᵢ (step S27). The relation extraction and prediction unit 33 exits the relation extraction and prediction processing.

[Effects of Embodiment 1]

According to Embodiment 1 above, the information processing apparatus including the machine learning device 1 and the prediction device 3 performs the following processing. For a first segment and a second segment included in a sentence, the information processing apparatus identifies a common ancestor node of a first node corresponding to the first segment and a second node corresponding to the second segment, which are two nodes included in the dependency tree generated from the sentence. The information processing apparatus encodes each node included in the dependency tree in accordance with a path from each of leaf nodes included in the dependency tree to the common ancestor node and thus acquires a vector of the common ancestor node. Based on the vector of the common ancestor node, the information processing apparatus encodes each of nodes included in the dependency tree in accordance with the path from the common ancestor node to the leaf nodes. Thus, the information processing apparatus may perform the sentence encoding based on outside of the shortest dependency path of the first segment and the second segment in the dependency tree.

According to Embodiment 1 above, the information processing apparatus aggregates information of the nodes to the common ancestor node along a path from each of leaf nodes to the common ancestor node and thus acquires a vector of the common ancestor node. Thus, because not only information of the shortest dependency path of the first segment and the second segment in the dependency tree but also information on each of nodes including a segment representing a relation outside the shortest dependency path are aggregated to the common ancestor node, the information processing apparatus may perform the sentence encoding based on the outside of the shortest dependency path. For example, the information processing apparatus is enabled to generate a vector properly including information on the outside of the shortest dependency path, which may improve the precision of the relation extraction between the first segment and the second segment.

According to Embodiment 1 above, the machine learning device 1 acquires a vector of a sentence from vectors representing encoding results of nodes. The machine learning device 1 inputs the vector of the sentence and a correct answer label corresponding to the vector of the sentence. The machine learning device 1 updates the machine learning model through machine learning based on a difference between a prediction result corresponding to the relation between the first segment and the second segment included in the sentence output by the machine learning model in accordance with the input and the correct answer label. Thus, the machine learning device 1 may generate a machine learning model that may extract the relation between the first segment and the second segment with high precision.

According to Embodiment 1, the prediction device 3 inputs a vector of another sentence to the updated machine learning model and outputs a prediction result corresponding to a relation between a first segment and a second segment included in the other sentence. Thus, the prediction device 3 may output the relation between the first segment and the second segment with high precision.

### Embodiment 2

It has been described that, according to Embodiment 1, the tree structure encoding unit 12 inputs a word to the LSTM and outputs an encode result vector encoded by the LSTM to the LSTM of the word positioned in the direction indicated by the parameter. However, without limiting thereto, the tree structure encoding unit 12 may input a word to the LSTM and output the encode result vector encoded by the LSTM and a predetermined position vector (positioning encoding: PE) of the word to the LSTM of the word positioned in the direction indicated by the parameter. The expression “predetermined position vector (PE)” refers to a dependency distance between a first segment and a second segment from which a relation is to be extracted in a sentence. Details of the predetermined position vector (PE) will be described below. [Configuration of Machine Learning Device According to Embodiment 2]

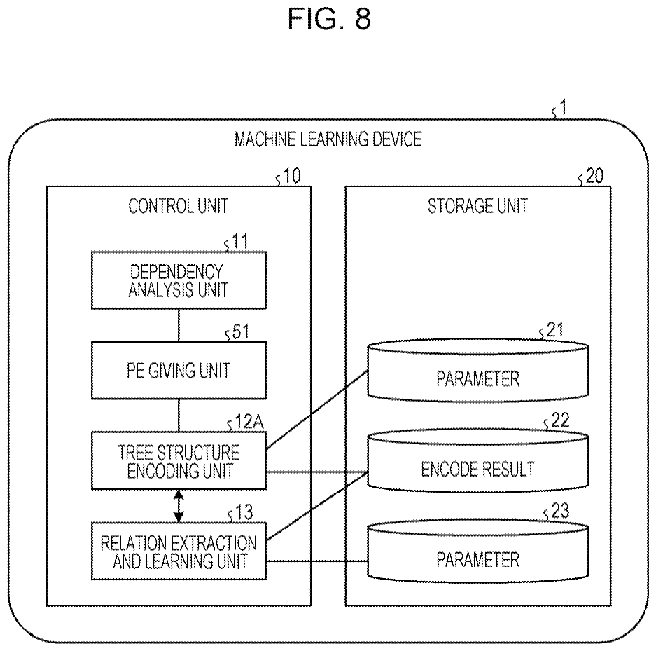

FIG. 8 is a functional block diagram illustrating a configuration of a machine learning device according to Embodiment 2. Elements of the machine learning device of FIG. 8 are designated with the same reference numerals as in the machine learning device 1 illustrated in FIG. 1, and the discussion of the identical elements and operation thereof is omitted herein. Embodiment 1 and Embodiment 2 are different in that a PE giving unit 51 is added to the control unit 10. Embodiment 1 and Embodiment 2 is further different in that the tree structure encoding unit 12 in the control unit 10 is changed to a tree structure encoding unit 12A.

The PE giving unit 51 provides each segment included in a sentence with a positional relation with a first segment included in the sentence and a positional relation with a second segment included in the sentence. For example, the PE giving unit 51 acquires a PE representing dependency distances to the first segment and the second segment of each segment by using a dependency tree having a tree structure. The PE is represented by (a,b) where a is a distance from the first segment and b is a distance from the second segment. As an example, the PE is represented by (Out) when a subject segment is not between the first segment and the second segment. The PE giving unit 51 gives the PE to each segment.

The tree structure encoding unit 12A encodes each segment by using the tree-structured LSTM of a tree converted to have a tree structure including dependencies of segments. For example, the tree structure encoding unit 12A uses dependencies of segments analyzed by the dependency analysis unit 11 and forms a converted dependency tree having a tree structure including the dependencies of segments. For a first segment and a second segment included in a sentence, the tree structure encoding unit 12A identifies a common ancestor node (LCA) of a first node corresponding to the first segment and a second node corresponding to the second segment, which are two nodes included in the converted dependency tree. The tree structure encoding unit 12A encodes each node included in the dependency tree along a path from each of leaf nodes included in the dependency tree to the LCA by using the parameter 21 and the PE and thus acquires a vector being an encoding result of the LCA. For example, the tree structure encoding unit 12A acquires the encoding result vector of the LCA by aggregating information including PEs of the nodes to the LCA along the path from each of leaf nodes to the LCA. Based on the encoding result vector of the LCA, the tree structure encoding unit 12A encodes each of the nodes included in the dependency tree along the path from the LCA to the leaf nodes by using the parameter 21 and the PEs. For example, the tree structure encoding unit 12A aggregates the information including PEs of the entire sentence to the LCA and then causes the aggregated information to reversely propagate to encode each node of the dependency tree.

By using the encoding result vectors of the nodes, the tree structure encoding unit 12A acquires a vector of the sentence.

[Configuration of Prediction Device According to Embodiment 2]

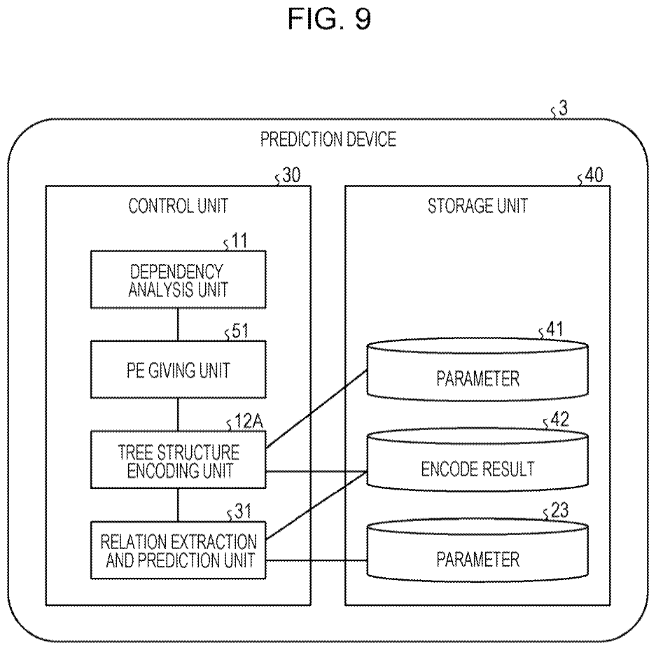

FIG. 9 is a functional block diagram illustrating a configuration of a prediction device according to Embodiment 2. Elements of the prediction device of FIG. 9 are designated with the same reference numerals as in prediction device 3 illustrated in FIG. 2, and the discussion of the identical elements and operation thereof is omitted herein. Embodiment 1 and Embodiment 2 are different in that a PE giving unit 51 is added to the control unit 10. Embodiment 1 and Embodiment 2 is further different in that the tree structure encoding unit 12 in the control unit 10 is changed to a tree structure encoding unit 12A. Because the PE giving unit 51 and the tree structure encoding unit 12A have the same configuration as those in the machine learning device 1 illustrated in FIG. 8, like numbers refer to like parts, and repetitive description on the configurations and operations are omitted.

[Example of Tree-Structured Encoding]

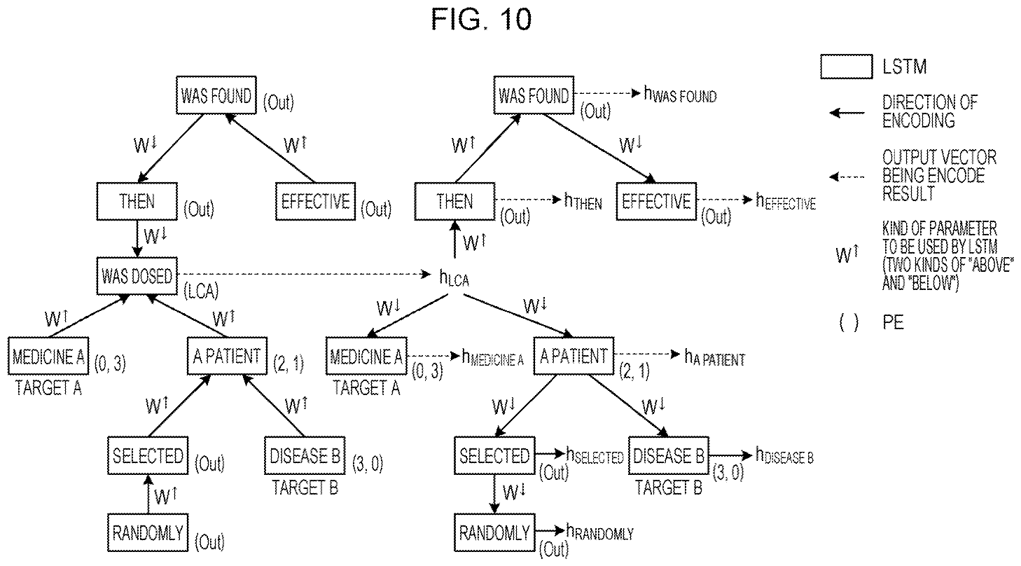

FIG. 10 illustrates an example of tree-structured encoding according to Embodiment 2. Suppose a case where a sentence “Medicine A was dosed to a randomly selected disease B patient, then, was found effective” is given and a relation (effective) between “Medicine A” and “disease B” is to be extracted (determined).

The left diagram of FIG. 10 illustrates a dependency tree having a tree structure in the sentence. The dependency tree is converted by the tree structure encoding unit 12A. For example, the tree structure encoding unit 12A uses dependencies of segments in the sentence analyzed by the dependency analysis unit 11 and converts them to a dependency tree having a tree structure including the dependencies of segments. Each of rectangular boxes in FIG. 10 represents an LSTM.

In addition, the PE giving unit 51 acquires a PE representing dependency distances to “Medicine A” and “disease B” for each segment by using the dependency tree having a tree structure and gives the acquired PE to the segment. PE is indicated on the right side of each LSTM. The PE of “Medicine A” is (0,3). For example, the distance from “Medicine A” is “0” because “Medicine A” is itself. The distance from “disease B” is “3” because there are “a patient”→“was dosed”→“Medicine A” about “disease B” as “0”. The PE of “a patient” is (2,1). For example, the distance from “Medicine A” is “2” because there are “was dosed”→“a patient” about “Medicine A” as “0”. The distance from “disease B” is “1” about “disease B” as “0”. The PE of “disease B” is (3,0). For example, the distance from “Medicine A” is “3” because there are “was dosed”→“a patient”→“disease B” about “Medicine A” as “0”. The distance from “disease B” is “0” because “disease B” is itself. The PEs of “selected” and “randomly” are “Out” because they are not between “Medicine A” and “disease B”. Also, the PEs of “then” and “was found” are “Out” because they are not between “Medicine A” and “disease B”.

For “Medicine A” and “disease B” included in the sentence, the tree structure encoding unit 12A identifies a common ancestor node (LCA) of the node corresponding to “Medicine A” and the node corresponding to “disease B”, which are two nodes included in the converted dependency tree. The identified LCA is a node corresponding to “was dosed”.

The tree structure encoding unit 12A encodes each node included in the dependency tree along a path from each of leaf nodes included in the dependency tree to the LCA by using the parameter 21 and the PE and thus acquires a vector being the encoding result of the LCA. For example, the tree structure encoding unit 12A aggregates information including PEs of the nodes to the LCA along the path from each of leaf nodes to the LCA. In the left diagram, the leaf nodes are the nodes corresponding to “Medicine A”, “randomly”, “disease B”, and “effective”.

As illustrated in the left diagram, the tree structure encoding unit 12A inputs “Medicine A” to the LSTM. The tree structure encoding unit 12A outputs a vector coupling an encode result (vector) encoded by the LSTM and the PE (0,3) to the LSTM of “was dosed” (LCA) positioned “above” indicated by the parameter.

The tree structure encoding unit 12A inputs “randomly” to the LSTM. The tree structure encoding unit 12A outputs a vector coupling an encode result (vector) encoded by the LSTM and the PE (Out) to the LSTM of “selected” positioned “above” indicated by the parameter.

The tree structure encoding unit 12A inputs “selected” and the vector from “randomly” to the LSTM. The tree structure encoding unit 12A outputs a vector coupling an encode result (vector) encoded by the LSTM and the PE (Out) to the LSTM of “a patient” positioned “above” indicated by the parameter.

The tree structure encoding unit 12A inputs “disease B” to the LSTM. The tree structure encoding unit 12A outputs a vector coupling an encode result (vector) encoded by the LSTM and the PE (3,0) to the LSTM of “a patient” positioned “above” indicated by the parameter.

The tree structure encoding unit 12A inputs “a patient”, the vector from “selected” and the vector from “disease B” to the LSTM. The tree structure encoding unit 12A outputs a vector coupling an encode result (vector) encoded by the LSTM and the PE (2,1) to the LSTM of “was dosed” (LCA) positioned “above” indicated by the parameter.

On the other hand, the tree structure encoding unit 12A inputs “effective” to the LSTM. The tree structure encoding unit 12A outputs a vector coupling an encode result (vector) encoded by the LSTM and the PE (Out) to the LSTM of “was found” positioned “above” indicated by the parameter.

The tree structure encoding unit 12A inputs “was found” and the vector from “effective” to the LSTM. The tree structure encoding unit 12A outputs a vector coupling an encode result (vector) encoded by the LSTM and the PE (Out) to the LSTM of “then” positioned “below” indicated by the parameter.

The tree structure encoding unit 12A inputs “then” and the vector from “was found” to the LSTM. The tree structure encoding unit 12A outputs a vector coupling an encode result (vector) encoded by the LSTM and the PE (Out) to the LSTM of “was dosed” (LCA) positioned “below” indicated by the parameter.

The tree structure encoding unit 12A inputs “was dosed”, the vector from “then”, the vector from “Medicine A”, and the vector from “a patient” to the LSTM. The tree structure encoding unit 12A acquires the encode result (vector) encoded by the LSTM as the encode result (vector) of the LCA. For example, the tree structure encoding unit 12A aggregates information of the nodes to the LCA along the path from each of leaf nodes to the LCA.

After that, based on the encode result (vector) of the LCA, the tree structure encoding unit 12A encodes each of the nodes included in the dependency tree along the path from the LCA to the leaf nodes by using the parameter 21 and PEs. For example, the tree structure encoding unit 12A aggregates information of the entire sentence to the LCA and then causes the information including the aggregated PEs to reversely propagate to encode each node of the dependency tree.

As illustrated in the right diagram, suppose that the encode result (vector) of LCA is h_{LCA}. The tree structure encoding unit 12A outputs h_{LCA} to the LSTMs of “Medicine A” and “a patient” positioned “below” indicated by the parameters toward the leaf nodes. The tree structure encoding unit 12A outputs h_{LCA} to the LSTM of “then” positioned “above” indicated by the parameter toward the leaf node.

The tree structure encoding unit 12A inputs “Medicine A” and h_{LCA} to the LSTM. The tree structure encoding unit 12A outputs h_{Medicine A} that is the encode result (vector) encoded by the LSTM.

The tree structure encoding unit 12A inputs “a patient” and h_{LCA} to the LSTM. The tree structure encoding unit 12A outputs h_{a patient} as the encode result (vector) encoded by the LSTM. The tree structure encoding unit 12A outputs the vector coupling h_{a patient} and PE(2,1) to the LSTMs of “selected” and “disease B” positioned “below” indicated by the parameters.

The tree structure encoding unit 12A inputs “selected” and the vector from “a patient” to the LSTM. The tree structure encoding unit 12A outputs h_{selected} as the encode result (vector) encoded by the LSTM. The tree structure encoding unit 12A outputs the vector coupling h_{selected} and PE(Out) to the LSTM of “randomly” positioned “below” indicated by the parameter.

The tree structure encoding unit 12A inputs “randomly” and the vector from “selected” to the LSTM. The tree structure encoding unit 12A outputs h_{randomly} as the encode result (vector) encoded by the LSTM.

The tree structure encoding unit 12A inputs “disease B” and the vector from “a patient” to the LSTM. The tree structure encoding unit 12A outputs h_{disease B} as the encode result (vector) encoded by the LSTM.

On the other hand, the tree structure encoding unit 12A inputs “then” and h_{LCA} to the LSTM. The tree structure encoding unit 12A outputs hₜₕₑₙ as the encode result (vector) encoded by the LSTM. The tree structure encoding unit 12A outputs the vector coupling hₜₕₑₙ and PE(Out) to the LSTM of “was found” positioned “above” indicated by the parameter.

The tree structure encoding unit 12A inputs “was found” and the vector from “then” to the LSTM. The tree structure encoding unit 12A outputs h_{was found} as the encode result (vector) encoded by the LSTM. The tree structure encoding unit 12A outputs a vector coupling h_{was found} and PE(Out) to the LSTM of “effective” positioned “below” indicated by the parameter.

The tree structure encoding unit 12A inputs “effective” and the vector from “was found” to the LSTM. The tree structure encoding unit 12A outputs h_{effective} as the encode result (vector) encoded by the LSTM.

From the vectors indicating the encode results of the nodes, the tree structure encoding unit 12A acquires a vector of the sentence. The tree structure encoding unit 12A may acquire a vector h_{sentence} of the sentence as follows. h_{sentence}=[h_{Medicine A};h_{randomly};h_{selected};h_{disease B};h_{a patient};h_{was dosed};hₜₕₑₙ;h_{effective};h_{was found};]

Thus, the tree structure encoding unit 12A clearly indicates a vector representing each word by adding a positional relation (PE) with respect to targets (“Medicine A” and “disease B”) thereto so that the handling may be changed between important information within the SP and information that is not important. As a result, the tree structure encoding unit 12A may encode a word with high precision based on whether the word is related to the targets or not. Hence, the tree structure encoding unit 12A may encode the sentence with high precision based on outside of the SP of “Medicine A” and “disease B” in the dependency tree.

[Effects of Embodiment 2]

According to Embodiment 2 above, the tree structure encoding unit 12A includes processing of aggregating information including a positional relation with a first node and a positional relation with a second node among nodes to a common ancestor node along a path from each of leaf nodes to the common ancestor node. Thus, the tree structure encoding unit 12A may change the handling between an important node and a node that is not important with respect to the first node and the second node. As a result, the tree structure encoding unit 12A may encode a node with high precision based on whether the node is related to the first node and the second node or not.

[Others]

According to Embodiments 1 and 2, it has been described that the information processing apparatus including the machine learning device 1 and the prediction device 3 performs the following processing on a sentence in English. For example, it has been described that the information processing apparatus aggregates information of an entire sentence in English to a common ancestor node in a dependency tree of the entire sentence and encodes each node of the dependency tree by using the aggregated information. However, without limiting thereto, the information processing apparatus is applicable for a sentence in Japanese. For example, the information processing apparatus may aggregate information of an entire sentence in Japanese to a common ancestor node in a dependency tree of the entire sentence and encodes each node of the dependency tree by using the aggregated information.

The illustrated components of the machine learning device 1 and the prediction device 3 do not necessarily have to be physically configured as illustrated in the drawings. For example, the specific forms of distribution and integration of the machine learning device 1 and the prediction device 3 are not limited to those illustrated in the drawings, but all or part thereof may be configured to be functionally or physically distributed or integrated in given units in accordance with various loads, usage states, and so on. For example, the tree structure encoding unit 12 may be distributed to an aggregation unit that aggregates information of nodes to the LCA and a reverse propagation unit that causes the information aggregated to the LCA to be reversely propagated. The PE giving unit 51 and the tree structure encoding unit 12 may be integrated as one functional unit. The storage unit 20 may be coupled via a network as an external device of the machine learning device 1. The storage unit 40 may be coupled via a network as an external device of the prediction device 3.

According to the embodiments above, the configuration has been described in which the machine learning device 1 and the prediction device 3 are separately provided. However, the information processing apparatus may be configured to include the machine learning processing by the machine learning device 1 and the prediction processing by the prediction device 3.

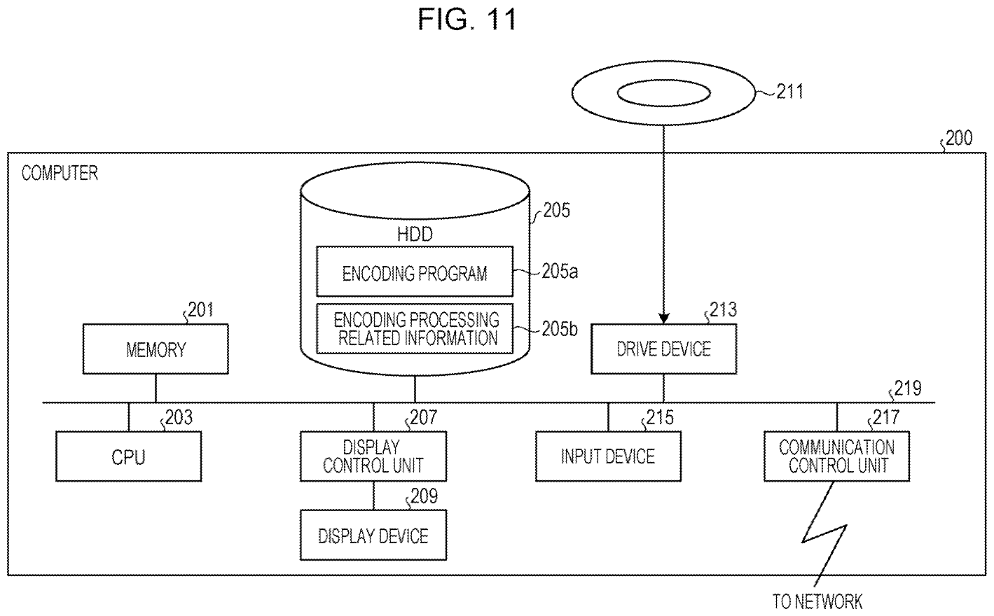

The various processes described in the embodiments above may be implemented as a result of a computer such as a personal computer or a workstation executing a program prepared in advance. Hereinafter, a description is given of an example of the computer that executes an encoding program for implementing functions similar to the functions of the machine learning device 1 and the prediction device 3 illustrated in FIG. 1. An encoding program for implementing functions similar to the functions of the machine learning device 1 will be described as an example. FIG. 11 illustrates an example of a computer that executes the encoding program.

As illustrated in FIG. 11, a computer 200 includes a CPU 203 that performs various kinds of arithmetic processing, an input device 215 that receives input of data from a user, and a display control unit 207 that controls a display device 209. The computer 200 further includes a drive device 213 that reads a program or the like from a storage medium 211, and a communication control unit 217 that exchanges data with another computer via a network. The computer 200 further includes a memory 201 that temporarily stores various types of information and a hard disk drive (HDD) 205. The memory 201, the CPU 203, the HDD 205, the display control unit 207, the drive device 213, the input device 215, and the communication control unit 217 are coupled to one another via a bus 219.

The drive device 213 is, for example, a device for a removable disk 210. The HDD 205 stores an encoding program 205a and encoding processing related information 205b.

The CPU 203 reads the encoding program 205a to deploy the encoding program 205a in the memory 201 and executes the encoding program 205a as processes. Such processes correspond to the functional units of the machine learning device 1. The encoding processing related information 205b corresponds to the parameter 21, the encode result 22 and the parameter 23. For example, the removable disk 210 stores various kinds of information such as the encoding program 205a.

The encoding program 205a may not be necessarily stored in the HDD 205 from the beginning. For example, the encoding program 205a may be stored in a “portable physical medium” such as a flexible disk (FD), a compact disk read-only memory (CD-ROM), a digital versatile disk (DVD), a magneto-optical disk, or an integrated circuit (IC) card inserted into the computer 200. The computer 200 may read the encoding program 205a from the portable physical medium and execute the encoding program 205a.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable storage medium storing an encoding program causing a computer to execute a process comprising:
identifying a common ancestor node of a first node corresponding to a first segment in a sentence and a second node corresponding to a second segment in the sentence, the first node and the second node being included in a dependency tree generated based on the sentence;
acquiring a vector of the common ancestor node by encoding each node included in the dependency tree in accordance with a path from each of leaf nodes included in the dependency tree to the common ancestor node; and
encoding, based on the vector of the common ancestor node, each of nodes included in the dependency tree in accordance with the path from the common ancestor node to the leaf nodes.

2. The storage medium according to claim 1,
wherein the processing of acquiring the vector of the common ancestor node includes processing of aggregating information of nodes to the common ancestor node along a path from each of leaf nodes to the common ancestor node and thus acquiring the vector of the common ancestor node.

3. The storage medium according to claim 2,
wherein the processing of aggregating includes processing of aggregating information including a positional relation with the first node and a positional relation with the second node among nodes to the common ancestor node along a path from each of leaf nodes to the common ancestor node.

4. The storage medium according to claim 1, wherein
a vector of the sentence is acquired from vectors representing encoding results of the nodes included in the dependency tree, and
input of the vector of the sentence and a correct answer label corresponding to the vector of the sentence is received, and, through machine learning based on a difference between a prediction result corresponding to a relation between the first segment and the second segment included in the sentence to be output by the machine learning model in accordance with the input and the correct answer label, the machine learning model is updated.

5. The storage medium according to claim 4,
wherein a vector of another sentence is input to the updated machine learning model, and a prediction result corresponding to a relation between a first segment and a second segment included in the another sentence is output.

6. An information processing apparatus comprising:
a memory, and
a processor coupled to the memory and configured to:
identify a common ancestor node of a first node corresponding to a first segment in a sentence and a second node corresponding to a second segment in the sentence, the first node and the second node being included in a dependency tree generated based on the sentence;
acquire a vector of the common ancestor node by encoding each node included in the dependency tree in accordance with a path from each of leaf nodes included in the dependency tree to the common ancestor node; and
encode, based on the vector of the common ancestor node, each of nodes included in the dependency tree in accordance with the path from the common ancestor node to the leaf nodes.

7. A computer-implemented method for encoding a sentence comprising:
identifying a common ancestor node of a first node corresponding to a first segment in the sentence and a second node corresponding to a second segment in the sentence, the first node and the second node being included in a dependency tree generated based on the sentence;
acquiring a vector of the common ancestor node by encoding each node included in the dependency tree in accordance with a path from each of leaf nodes included in the dependency tree to the common ancestor node; and
encoding, based on the vector of the common ancestor node, each of nodes included in the dependency tree in accordance with the path from the common ancestor node to the leaf nodes.
